(19) United States
(12) Patent Application Publication
(10) Pub. No.: US 2017/0164607 A1
(43) Pub. Date: Jun. 15, 2017

(54) METHODS OF EXTENDING THE SHELF LIFE OF SUGARCANE STEM SECTIONS AND COATED SUGARCANE STEM SECTIONS

(71) Applicants: Nicholas D. Nicholas, Vero Beach, FL (US); Henry AGBAJE, Greensboro, NC (US); Clark Lovelady, Vero Beach, FL (US); Xuebing ZHANG, Greensboro, NC (US); Syngenta Participations AG, Basel (CH)

(72) Inventors: Nicholas D. Polge, Vero Beach, FL (US); Henry AGBAJE, Greensboro, NC (US); Clark Nolan Lovelady, Vero Beach, FL (US); Xuebing ZHANG, Greensboro, NC (US)

(73) Assignee: Syngenta Participations AG, Basel (CH)

(21) Appl. No.: 15/126,453

(22) PCT Filed: Mar. 9, 2015
(86) PCT No.: PCT/US15/19361

§ 371 Date: Sep. 15, 2016

Related U.S. Application Data
(60) Provisional application No. 61/952,401, filed on Mar. 13, 2014.

(51) Int. Cl. A01N 3/00; A01H 5/04; A01N 59/16

(52) U.S. Cl. CPC A01N 3/00; A01N 59/16; A01H 5/04

(57) ABSTRACT

Methods of extending the shelf life and vitality of sugarcane stem sections are disclosed. Coated sugarcane stem sections are also disclosed. In one exemplary embodiment, the method of extending the shelf life and vitality of a sugarcane stem section comprises coating at least a portion of the sugarcane stem section with a coating composition comprising at least one additive selected from: silver nitrate, silver acetate, silver thiosulfate, potassium ferrocyanide, and potassium ferricyanide so as to form a coated sugarcane stem section.

### TECHNICAL FIELD

The present invention relates to methods of extending the shelf life of sugarcane stem sections and coated sugarcane stem sections.

### BACKGROUND

Sugarcane is a gramineous plant of commercial importance for a variety of reasons. For example, sugarcane is used for the production of sugar, Falernum, molasses, rum, cachaça (the national spirit of Brazil), and ethanol for fuel. Further, the biomass that remains after sugarcane crushing can also be used in furnaces and boilers.

Most commercial sugarcane is grown from stem sections (also known as cane cuttings or parts of a stalk or culms or carretels or seedlings). Stem sections may be produced from the stem of a sugarcane plant in any number of ways. For example, they may be formed manually or by a variety of machines. The resulting stem sections usually include one or more nodes per stem section. The term “node” refers to the part of the stem of a plant from which a leaf, branch, or aerial root grows.

After stem sections are planted, buds (or gemmas) may emerge at the position of each node. Buds may then grow to yield the crop plant. However, emergence rate or germination rate, or the rate at which nodes bud to yield crop plants is sometimes poor in sugarcane, especially when the stem section has experienced stress prior to planting. Stem section stress, for example, may be in the form of prolonged storage (i.e., up to and greater than 7 days) prior to planting.

Typical shelf life in Brazil at ambient temperature is 2-4 days or less. The germination of sugarcane stem sections decreases rapidly thereafter (i.e. the 2-4 days or less). Some attempts to improve shelf life of sugarcane stem sections have been limited to storage at cold temperatures; however, such attempts, at best, extended the “2-4 days or less” by 2-4 days. In some cases, for example, bulk bag storage at temperatures of from 13° C. to 16° C., the shelf life of sugarcane stem sections may have been from about 7 to 10 days. However, the vigor/vitality of the sugarcane stem sections decreased rapidly under the conditions used in the above-described attempts.

There exists a need in the art for sugarcane stem sections that possess an improved shelf life, while maintaining a desired degree of vigor and vitality.

### SUMMARY

The present invention addresses the need in the art for sugarcane stem sections that possess the combination of both (i) an improved shelf life, and (ii) a desired degree of vigor and vitality. Accordingly, the present invention is directed to methods of extending the shelf life and vitality of sugarcane stem sections. In one exemplary embodiment, the method of extending the shelf life and vitality of a sugarcane stem section comprises: coating at least a portion of the sugarcane stem section with a coating composition comprising at least one additive selected from: silver nitrate, silver acetate, silver thiosulfate, potassium ferrocyanide, and potassium ferricyanide, so as to form a coated sugarcane stem section. Methods of extending the shelf life and vitality of sugarcane stem sections may further comprise steps such as drying the coated sugarcane stem section at a drying temperature to form a dried coated sugarcane stem section; and storing the dried coated sugarcane stem section for a storage time period in a storage container at a storage temperature.

The present invention is also directed to methods increasing an average germination rate of sugarcane stem sections. In one exemplary embodiment, the method of increasing an average germination rate of sugarcane stem sections comprises: forming coated sugarcane stem sections using the herein disclosed methods. In some embodiments of the present invention, the coated sugarcane stem sections have (i) an average germination rate greater than an average germination rate of control uncoated sugarcane stem sections, (ii) an average germination rate of greater than 90% after storage for 21 days, or (iii) both (i) and (ii).

The present invention is further directed to coated sugarcane stem sections that possess the combination of both (i) an improved shelf life, and (iii) a desired degree of vigor and vitality. In one exemplary embodiment, the coated sugarcane stem section comprises a sugarcane stem section at least partially coated with a coating comprising at least one additive selected from: silver nitrate, silver acetate, silver thiosulfate, potassium ferrocyanide, and potassium ferricyanide. In some desired embodiments, the coated sugarcane stem section comprises a sugarcane stem section at least partially coated with a coating comprising silver nitrate.

The present invention is further directed to methods of growing sugarcane plants. In one exemplary embodiment, the method of growing sugarcane plants comprises: planting one or more coated sugarcane stem sections, wherein each coated sugarcane stem section comprises a sugarcane stem section at least partially coated with a coating comprising at least one additive selected from: silver nitrate, silver acetate, silver thiosulfate, potassium ferrocyanide, and potassium ferricyanide.

The present invention is even further directed to methods of increasing the vitality of sugarcane plants formed from sugarcane stem sections. In one exemplary embodiment, the methods of increasing the vitality of sugarcane plant formed from sugarcane stem sections comprises: forming coated sugarcane stem sections using the herein disclosed methods; and planting the coated sugarcane stem sections in a growing medium. In some embodiments, the sugarcane plants resulting from the coated sugarcane stem sections have an average shoot height at least greater than an average shoot height of control sugarcane plants resulting from planting one or more control uncoated sugarcane stem sections.

The above summary was intended to summarize certain embodiments of the present disclosure. Methods, compositions and products will be set forth in more detail, along with examples demonstrating efficacy, in the figures and detailed description below. It will be apparent, however, that the detailed description is not intended to limit the present invention, the scope of which should be properly determined by the appended claims.

### BRIEF DESCRIPTION OF THE FIGURES

The present invention is further described with reference to the appended figures, wherein:

FIG. 1 depicts exemplary coated sugarcane stem sections within a storage container;

FIGS. 2-7 graphically depict germination rates of coated and control uncoated sugarcane stem sections after storage in air-tight jars for a period of time;

FIGS. 8-9 graphically depict germination rates of coated and control uncoated sugarcane stem sections after storage in gas-permeable storage containers as shown in FIG. 1 for a period of time, and shoot heights of sugarcane plants produced via the coated and control uncoated sugarcane stem sections;

FIG. 10 depict sugarcane plants produced via coated sugarcane stem sections of the present invention and control uncoated sugarcane stem sections; and

FIGS. 11-12 graphically depict germination rates of coated and control uncoated sugarcane stem sections after storage in gas-permeable storage containers as shown in FIG. 1 for a period of time up to 42 days, and shoot heights of sugarcane plants produced via the coated and control uncoated sugarcane stem sections.

### DETAILED DESCRIPTION

The present invention is directed to methods of extending the shelf life and vitality of sugarcane stem sections. The disclosed methods of extending the shelf life and vitality of a sugarcane stem section comprise: coating at least a portion of the sugarcane stem section with a coating composition comprising at least one additive selected from: silver nitrate, silver acetate, silver thiosulfate, potassium ferrocyanide, and potassium ferricyanide, so as to form a coated sugarcane stem section. In desired embodiments, the methods of extending the shelf life and vitality of a sugarcane stem section comprise: coating at least a portion of the sugarcane stem section with a coating composition comprising at least one additive comprising silver nitrate.

The coating step may comprise coating cut ends of the sugarcane stem section with a given coating composition, or coating all of the sugarcane stem section with the given coating composition. Further, the coating step may comprise at least one method step chosen from dipping, spraying, brushing and smearing. Typically, the coating step comprises dipping the sugarcane stem section in the coating composition for a time period. Typically, the time period (i.e., dwell time in a dipping bath) is less than one minute.

Sugarcane stem sections used in the present invention may comprise any sugarcane stem section having a desired length, diameter, and number of nodes. Typically, each of the sugarcane stem sections used in the present invention comprises one or more nodes along a given sugarcane stem section although multi-node sugarcane stem sections may also be used. In some embodiments, the sugarcane stem sections used in the present invention each comprise a single node along the sugarcane stem section. In one embodiment the length of stem section is generally from about 0.5 to about 20 cm in length, from about 2 to about 12 cm, from about 3 to about 8 cm, from about 3.5 to 5.5 cm, from about 3.5 to 4.5 cm, about 4-5 cm, from about 0.5 to 2.5 cm, and from about 0.5 to 1.5 cm, in length.

Each of the above-mentioned additives may be independently coated onto a given sugarcane stem section at an independently desired concentration. In other words, a first additive, such as silver nitrate, can be coated onto a given sugarcane stem section at a concentration of about 1200 μMol, while a second additive, such as silver acetate, can be coated onto the same sugarcane stem section at a concentration of about 300 μMol.

Typically, a given costing composition comprises one or more of the above-described additives, wherein each additive is independently present at a concentration of up to about 2400 μMol although the present invention is not limited to such concentrations. In some embodiments, the coating composition comprises one or more of the above-described additives, wherein each additive is independently present at a concentration of from about 200 to about 1200 μMol. In other embodiments, the coating composition comprises one or more of the above-described additives, wherein each-additive, is independently present at a concentration of from about 300 to about 600 μMol. In other embodiments, the coating composition comprises one or more of the above-described additives, wherein each additive is independently present at a concentration of about 600 μMol.

Typically, a given coating composition comprises a single additive at a concentration ranging from about 200 to about 1200 μMol. As discussed above, in some desired embodiments, a given coaling composition comprises a single additive in the form of silver nitrate at a concentration ranging from about 200 to about 1200 μMol (or any concentration or range of concentration values therebetween in increments of 0.1 μMol).

Coating compositions used in the present invention may include, in addition to one or more of the above-mentioned additives, a fungicide, an insecticide, or both. When present, suitable fungicides include but are not limited to acycloamino acid fungicides, aliphatic nitrogen fungicides, amide fungicides, anilide fungicides, antibiotic fungicides, aromatic fungicides, arsenical fungicides, aryl phenyl ketone fungicides, benzamide fungicides, benzanilide fungicides, imidazole fungicides, benzothiazole fungicides, botanical fungicides, bridged diphenyl fungicides, carbamate fungicides, carbamolate fungicides, conazole fungicides, copper fungicides, dicarboximide fungicides, dinitrophenol fungicides, dithiocarbamate fungicides, dithiolane fungicides, furamide fungicides, furanilide fungicides, hydrazide fungicides, imidazole fungicides, mercury fungicides, morpholine fungicides, organophosphorous fungicides, organotin fungicides, oxathiin fungicides, oxazole fungicides, phenylsulfamide fungicides, polysulfide fungicides, pyrazole fungicides, pyridine fungicides, pyrimidine fungicides, pyrrole fungicides, quaternary ammonium fungicides, quinoline fungicides, quinone fungicides, quinoxaline fungicides, strobilurin fungicides, sulfonanilide fungicides, thiadiazole fungicides, thiazole fungicides, thiazolidine fungicides, thiocarbamate fungicides, thiophene fungicides, triazine fungicides, triazole fungicides, triazolopyrimidine fungicides, urea fungicides, valinanide fungicides, and zinc fungicides. Particularly suitable are fungicides such fludioxonil, thiabendazole, metataxyl and mefenoxam, strobilurin fungicides such azoxystrobin, picoxystrobin, pyraclostrobin, pyraoxystrobin, and kresoxim-methyl SDHI fungicides such as boscalid, sedaxane, penthiopyrad, penflufen, isopyrazam, furametpyr, fluxapyroxad, bixafen, thifluzamide, carboxin, oxycarboxin, fenfuram, fluopyram, benoldanil, flutolanil, and mepronil. When present, suitable insecticides include, but are not limited to benzoylureas, carbamates, chloronicotinyls, diacylhydrazines, diamides, fiproles, macrolides, nitroimines, nitromethylenes, organochlorines, organophosphates, organosilicons, organolins, phenylpyrazoles, phosphoric esters, pyrethroids, spinosyns, tetramic acid derivatives and tetronic acid derivative. Particularly suitable insecticides include neonicotinoid insecticides such as thiamethoxam, clothianidin, thiacloprid, and imidacloprid, abamectin, and diamide insecticides such as chlorantranilprole, cyantraniliprole, cyclaniliprole, and flubendiamide. In addition to fungicides and insecticides, the coating compositions may comprise, one or more of the following composition components such as binders, latex, antimicrobials, preservatives, fertilizers, plant hormones, and water.

When present, each of the above-mentioned additional composition components (other than water) may be present in any amount but are typically each independently present in amounts ranging from greater than 0 to about 20 wt % of the total weight of the coating composition.

The disclosed methods of extending the shelf life and vitality of sugarcane stem sections may further comprise drying the coated sugarcane stem section at a drying temperature to form a dried coated sugarcane stem section. Typically, the drying step comprises drying the coated sugarcane stem sections at a drying temperature, wherein the drying temperature is room temperature. Un some embodiments, the drying temperature ranges from about 10° C. to about 55° C.

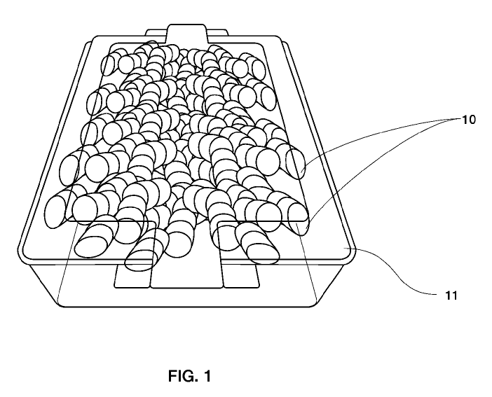

The dried coated sugarcane stem section, as shown dried coated sugarcane stem section 10 in FIG. 1, may comprise any amount of dried coating composition thereon. Typically, dried coated sugarcane stem sections of the present invention comprise from about 0.01 to about 10.0 g of coating composition thereon. As used herein, the term “dried” means dry to the touch, for example, when touched with water sensitive paper.

The disclosed methods of extending the shelf life and vitality of sugarcane stem sections may even further comprise storing the dried coated sugarcane stem sections for a storage time period in a storage container at a storage temperature. The storage time period may be six weeks or longer. Typically, the storage time period is up to about four weeks. In some embodiments, the storage time period is from about seven days to about 21 days. The storage period will vary depending on a number of factors including, but not limited to, the production needs and scheduling of processors that need the coated sugarcane stem sections of the present invention.

The dried coated sugarcane stem section may be stored in a variety of containers. In some embodiments, the dried coated sugarcane stem sections are stored in air-tight containers such as jars. In other embodiments, the dried coated sugarcane stem sections are stored in storage containers that allow air transfer between an interior of the storage container and an exterior of the storage container. Such a storage container is shown in FIG. 1 as storage container 11.

The storage temperature may vary depending on a number of factors including, but not limited to, the manufacturing location, the time of year, the period of storage, etc. In some embodiments, the storage temperature is room temperature, whatever the room temperature is at a given location. In other embodiments, the storage temperature ranges from about 10° C. to about 40° C. In other embodiments, the storage temperature ranges from about 20° C. to about 35° C.

In some embodiments, the storage step further comprises minimizing light exposure of the dried coated sugarcane stem section during storage. As used herein, the phrase “minimizing light exposure” refers to limiting exposure to natural light and/or artificial light to less than 3 hours a day, desirably, less than 1 hour a day.

The present invention is also directed to methods of increasing an average germination rate of sugarcane stem sections. In one exemplary embodiment, the method of increasing an average germination rate of sugarcane stem sections comprises forming coated sugarcane stem sections using any one of the above-disclosed methods. As discussed below in the example section, in some embodiments of the present invention, the coated sugarcane stem sections have (i) an average germination rate greater than an average germination rate of control uncoated sugarcane stem sections, (ii) an average germination rate of greater than 90% after storage for 21 days, or (iii) both (i) and (ii).

The present invention is further directed to coated sugarcane stem sections formed by the methods disclosed herein. The coated sugarcane stem sections, formed by the methods disclosed herein, provide a number of advantages over uncoated sugarcane stem sections. In some embodiments, a plurality of the coated sugarcane stem sections has an average germination rate greater than an average germination rate of a plurality of control uncoated sugarcane stem sections. As used herein, the phrase “control uncoated sugarcane stem sections” is used to describe sugarcane stem sections that are treated identically to a corresponding set of coated sugarcane stem sections except that the “control uncoated sugarcane stem sections” are not coated. In other words, the “control uncoated sugarcane stem sections” are cut, and stored for a storage time period in a storage container at a storage temperature that is identical to corresponding coated sugarcane stem sections that have been cut, coated, and stored for an identical storage time period in an identical storage container at an identical storage temperature.

In some embodiments, a plurality of the coated sugarcane stem section, formed by the methods disclosed herein, has an average germination rare of greater than 80% after storage for 21 days. In other embodiments, a plurality of the coated sugarcane stem section, formed by the methods disclosed herein, has an average germination rate of greater than 90% after storage for 21 days.

In addition, a plurality of the coated sugarcane stem section, formed by the methods disclosed herein, produce sugarcane plants having an average shoot height as greater than an average shoot height of sugarcane plants resulting from planting control uncoated sugarcane stem sections.

The present invention is even further directed to methods of growing sugarcane plants. In one exemplary embodiment, the method of growing sugarcane plants comprises: planting (i) one or more coated sugarcane stem sections formed by the methods of the present invention in a growing medium.

The present invention is even further directed to methods of increasing the vitality of sugarcane plants formed from sugarcane stem sections. In one exemplary embodiment, the method of increasing the vitality of sugarcane plants formed from sugarcane stem sections comprises: forming coated sugarcane stem sections using any one of the herein-described methods; and planting one or more coated sugarcane stem sections formed by any one of the herein-described methods.

In methods of growing sugarcane plants and methods of increasing the vitality of sugarcane plants formed from sugarcane stem sections, the coated sugarcane stem sections may be stored up to 42 days (or more) prior to planting. In some embodiments, the coated sugarcane stem sections are stored for more than 10 days prior to planting. In other embodiments, the coated sugarcane stem sections are stored for from about 7 to about 21 days prior to planting.

The present invention is even further directed to sugarcane plants formed from coated sugarcane stem sections of the present invention. In some exemplary embodiments, the sugarcane plants resulting from coated sugarcane stem sections of the present invention (i.e., formed via the above-described methods) have an average shoot height greater than an average shoot height of sugarcane plants resulting from planting one or more control uncoated sugarcane stem sections.

### OTHER EMBODIMENTS

### Methods of Extending Shelf Life and Vitality of Sugarcane Stem Sections

1. A method of extending shelf life and vitality of a sugarcane stem section, said method comprising: coating at least a portion of the sugarcane stem section with a coating composition comprising at least one additive selected from: silver nitrate, silver acetate, silver thiosulfate, potassium ferrocyanide, and potassium ferricyanide, so as to form a coated sugarcane stem section.

2. The method of embodiment 1, wherein said coating step comprises coating cut ends of the sugarcane stem section with the coating composition.

3. The method of embodiment 1 or 2, wherein said coating step comprises coating all of the sugarcane stem section with the coating composition.

4. The method of any one of embodiments 1 to 3, wherein the sugarcane stem section comprises one or more nodes along the sugarcane stem section.

5. The method of any one of embodiments 1 in 4, wherein the sugarcane stem section comprises a single node along the sugarcane stem section.

6. The method of any one of embodiments 1 to 5, wherein the coating composition comprises silver nitrate.

7. The method of any one of embodiments 1 to 6, wherein the coating composition comprises one or more additives, wherein each additive is independently present at a concentration of up so about 2400 μMol.

8. The method of any one of embodiments 1 to 7, wherein the coating composition comprises one or more additives, wherein each additive is independently present at a concentration of from about 200 to about 1200 μMol.

9. The method of any one of embodiments 1 to 8, wherein the coating composition comprises one or more additives, wherein each additive is independently present at a concentration of from about 300 to about 600 μMol.

10. The method of any one of embodiments 1 to 9, wherein the coating composition comprises one or more additives, wherein each additive is independently present at a concentration of about 600 μMol.

11. The method of any one of embodiments 1 to 10, wherein the coating composition further comprises a fungicide, an insecticide, or both.

12. The method of any one of embodiments 1 to 11, wherein said coating step comprises at least one method chosen from dipping, spraying, brushing and smearing.

13. The method of any one of embodiments 1 to 12, wherein said coating step comprises dipping the sugarcane stem section in the coating composition for a time period.

14. The method of embodiment 13, wherein the time period is less than one minute.

15. The method of any one of embodiments 1 to 14, further comprising: drying the coated sugarcane stem section at a drying temperature to from a dried coated sugarcane stem section.

16. The method of embodiment 15, wherein the drying temperature is room temperature.

17. The method of embodiment 13 or 16, wherein the drying temperature ranges from about 10° C. to about 55° C.

18. The method of any one of embodiments 15 to 17, wherein the dried coated sugarcane stem section comprises from about 0.01 to 100 μMol of silver nitrate, silver acetate, silver thiosulfate, potassium ferrocyanide, or potassium ferricyanide, or a combination thereof.

19. The method of any one of embodiments 15 to 18, further comprising: storing the dried coated sugarcane stem section for a storage time period in a storage container at a storage temperature.

20. The method of embodiment 19, wherein the storage time period is up to four weeks.

21. The method of embodiment 19 or 20, wherein the storage time period is from about seven days to about 21 days.

22. The method of any one of embodiments 19 to 21, wherein the storage container allows air transfer between an interior of the storage container and an exterior of the storage container.

23. The method of any one of embodiments 19 to 22, wherein the storage temperature is room temperature.

24. The method of any one of embodiments 19 to 23, wherein the storage temperature ranges from about 10° C. to about 40° C.

25. The method of any one of embodiments 19 to 24, wherein the storage temperature ranges from about 10° C. to about 30° C.

26. The method of any one of embodiments 19 to 25, wherein said storage step further comprises minimizing light exposure of the dried coated sugarcane stem section.

### Methods of Increasing an Average Germination Rate of Sugarcane Stem Sections

27. A method of increasing an average germination rate of sugarcane stem sections, said method comprising: forming coated sugarcane stem sections using the method of any one of embodiments 1 to 26.

### Coated Sugarcane Cutting Section

28. A coated sugarcane cutting section formed by the method of any one of embodiments 1 to 27.

29. The coated sugarcane cutting section of embodiment 28, wherein a plurality of said coated sugarcane cutting section has an average germination rate greater than an average germination rate of a plurality of control uncoated sugarcane stem sections.

30. The coated sugarcane cutting section of embodiment 28 or 29, wherein a plurality of said coated sugarcane cutting section has an average germination rate of greater than 80% after storage for 21 days.

31. The coated sugarcane cutting section of any one of embodiments 28 to 30, wherein a plurality of said coated sugarcane cutting section has an average germination rate of greater than 90% after storage for 21 days.

32. The coated sugarcane cutting section of any one of embodiments 28 to 31, wherein sugarcane plants resulting from the coated sugarcane stem sections have an average shoot height greater than an average shoot height of control sugarcane plants resulting from planting uncoated sugarcane stem sections.

### Methods of Growing Sugarcane Plants

33. A method of growing sugarcane plants comprising: planting (i) one or more coated sugarcane cutting sections formed by the method of any one of embodiments 1 to 27, (ii) the coated sugarcane stem section of any one of embodiments 28 to 32, or (iii) both (i) and (ii) in a growing medium.

### Methods of Increasing the Vitality of Sugarcane Plants

34. A method of increasing vitality of sugarcane plants formed from sugarcane stem sections, said method comprising: forming coated sugarcane stem sections using the method of any one of embodiments 1 to 27, and planting (i) one or more coated sugarcane stem sections formed by the method of any one of embodiments 1 to 27, (ii) the coated sugarcane stem section of any one of embodiments 28 to 32, or (iii) both (i) and (ii) in a growing medium.

35. The method of embodiment 33 or 34, wherein the coated sugarcane stem sections are stored up to 28 days prior to said planting step.

36. The method of any one of embodiments 33 to 35, wherein the coated sugarcane stem sections are stored for more than 10 days prior to said planting step.

37. The method of any one of embodiments 33 to 35, wherein the coated sugarcane stem sections are stored for from about 7 to 21 days prior to said planting step.

38. The method of any one of any one of embodiments 33 to 37, wherein the sugarcane plants resulting from the coated sugarcane stem sections have an average shoot height at greater than an average shoot height of control sugarcane plants resulting from planting one or more uncoated control sugarcane stem sections.

The following examples are for illustration only, and are in no way intended to limit the scope of the invention.

### EXPERIMENT 1

### Preparation, Storage and Planting of Coated Sugarcane Stem Sections

Sugarcane stem sections having a diameter of approximately 35-30 mm and a length of approximately 50 mm were generated (e.g., cut) from sugarcane stalks. Coating compositions comprising silver nitrate were prepared at various concentrations as shown in Table 1 below. One set of cut sugarcane stem section were dipped into the coating compositions for a dwell time of about 60 seconds, and subsequently allowed to dry at room temperature (i.e., about 30° C. Another set of control sugarcane stem sections were not coated and remained at room temperature (i.e., about 30° C.) (i.e., identified below as “No AgNO₃”). The coated and control uncoated sugarcane stem sections were then planted as is (i.e., these stem sections are identified below as “Day Zero”) or stored for a period of time up to three weeks in an air tight jar at room temperature (i.e., about 30° C.). Following storage, the coated and control uncoated sugarcane stem sections were then germinated in biochamber boxes. All coated and control uncoated sugarcane stem sections were germinated in a growth chamber and evaluated seven days after planting.

Results are summarized in Table 1 below.

TABLE 1
Average Plant Heights and Germination Rates

| | Avg. | | |
|---|---|---|---|
| Sample | Height [mm] | Avg. Germ [%] | Std. Dev. |
| Day Zero | 50.21 | 78.57 | 3.30 |
| No AgNO₃, 2 weeks | 48.29 | 61.90 | 20.20 |
| 200 μMol AgNO₃, 2 weeks | 51.38 | 83.33 | 3.36 |
| 400 μMol AgNO₃, 2 weeks | 39.66 | 52.38 | 47.14 |
| 600 μMol AgNO₃, 2 weeks | 40.79 | 61.90 | 53.90 |
| No AgNO₃, 3 weeks | 16.28 | 35.00 | 35.00 |
| 200 μMol AgNO₃, 3 weeks | 42.91 | 52.50 | 46.00 |
| 400 μMol AgNO₃, 3 weeks | 30.53 | 45.00 | 49.50 |
| 600 μMol AgNO₃, 3 weeks | 55.19 | 87.50 | 3.50 |

As shown in Table 1, the coaled sugarcane stem sections, showed increased germination at the 200 μmol concentration compared to the control “no AgNO₃” sample after 2 weeks of storage in an air-tight jar. After a weeks of storage in an air-tight jar, the AgNO₃ coated sugarcane stem section samples demonstrated improved germination and plant height over the control “non-AgNO₃” samples.

### EXPERIMENT 2

### Preparation, Storage and Planting of Coated Sugarcane Stem Sections

Sugarcane stem sections (i.e., two varieties: CP 88-1762 and CP00-1101) having a diameter of approximately 25-30 mm and a length of approximately 50 mm were cut and coated (or left uncoated) as described in Example 1 using coating compositions having AgNO₃ concentrations of 0, 300, 600 and 1200 μMol of AgNO₃. The coated and control uncoated sugarcane stem sections were then planted as is or stored for a period of time up to three weeks in one gallon air-tight jars (i.e., about 20 coated sugarcane stem sections per jar) at 16° C. Following storage, the coated and control uncoated sugarcane stem sections were then germinated in either biochamber boxes or potted soil trays (i.e., 5 jars each) or both. All coated and control uncoated sugarcane stem sections were planted and evaluated as follows: germination rates were taken in the biochamber boxes at 3, 5, and 7 days after removal from the jars: germination rates were taken in the potted trays at 10, 12, and 14 days after removal from the jars.

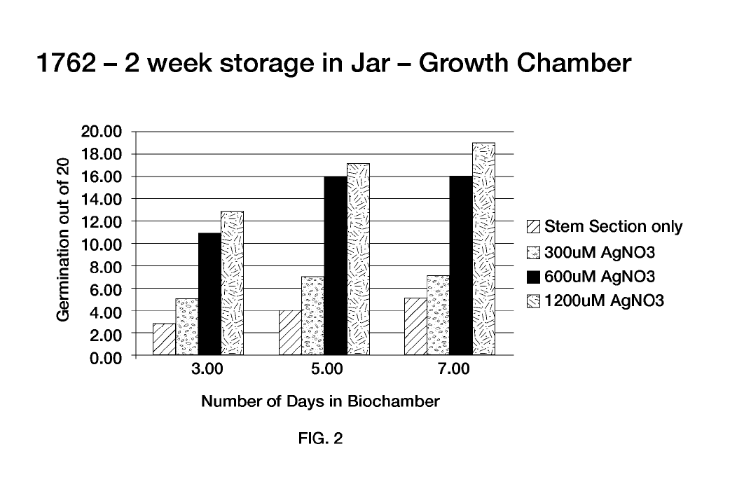

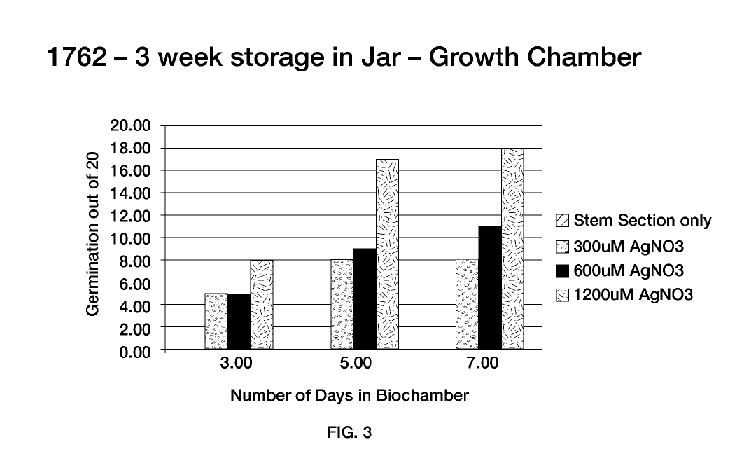

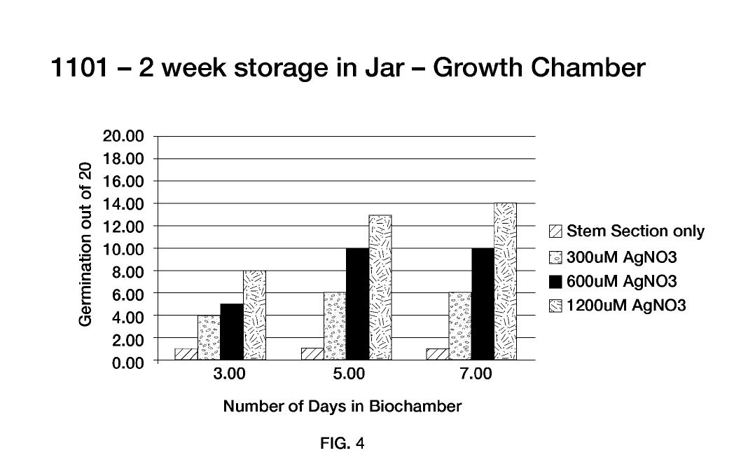

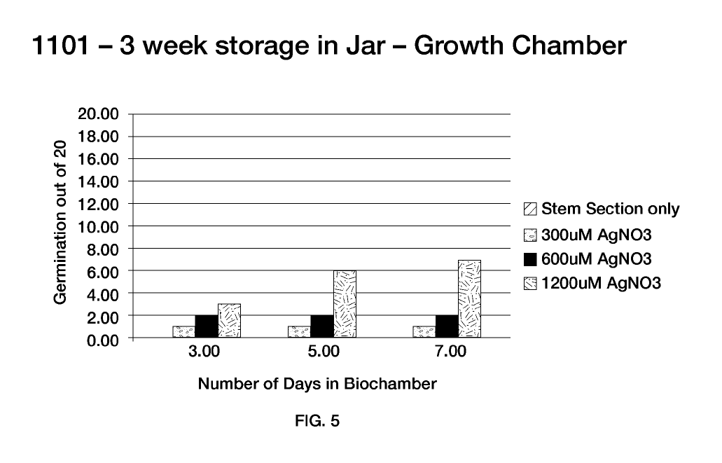

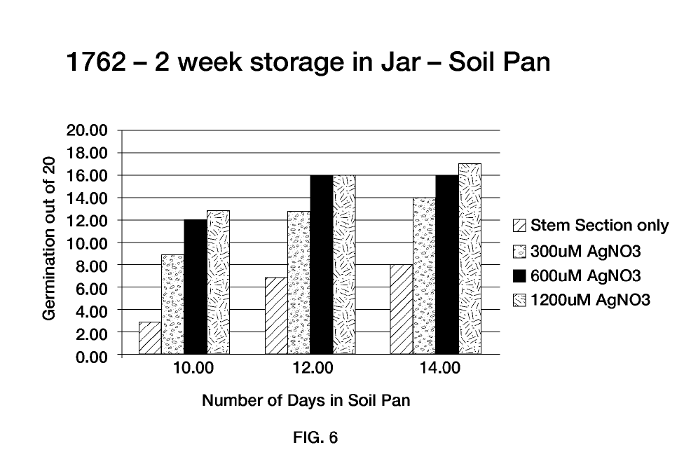

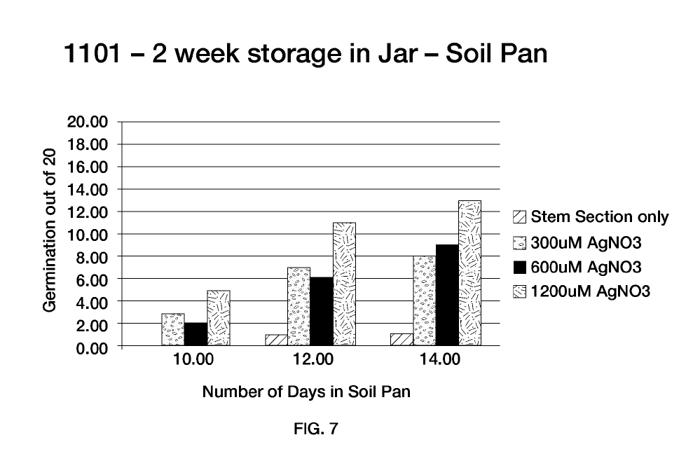

Results are shown in FIGS. 2-7 (note, the term “Plene” refers to a sugarcane stem section treated with a control coating/slurry composition). As shown in FIGS. 2-7, in all cases, the coating/slurry compositions containing AgNO3 applied to sugarcane stem sections showed improved germination rates when compared to the standard slurry applied sugarcane stem sections.

### EXPERIMENT 3

### Preparation, Storage and Planting of AgNO₃ Treated Sugarcane Stem Sections

Sugarcane stem sections of CP88-1762 having a diameter of approximately 25-30 mm and a length or approximately 50 mm were coated with a standard treatment slurry (designated control treatment) or treated with the standard treatment slurry which also contained AgNO₃ at a concentration of 600 μMol of AgNO₃ (designated as AgNO₃ treatment). The AgNO₃ treated and control sugarcane stem sections were then planted as is or stored for a period of time up to three weeks in storage containers as shown in FIG. 1 (i.e., storage containers that allow gas transfer between an interior and an exterior of the storage container) at storage temperatures ranging from 10° C. to 22° C. Following storage, the AgNO₃ treated and control sugarcane stem sections were planted in in trays of potting soil and placed in a greenhouse. The trays were watered daily to maintain moisture and the greenhouse maintained at 26/20 C maximum/minimum temperatures with 15 hours light. Growth and germination of all AgNO₃ treated and control sugarcane stem sections were evaluated at 7, 14, and 21 days after removal from storage containers.

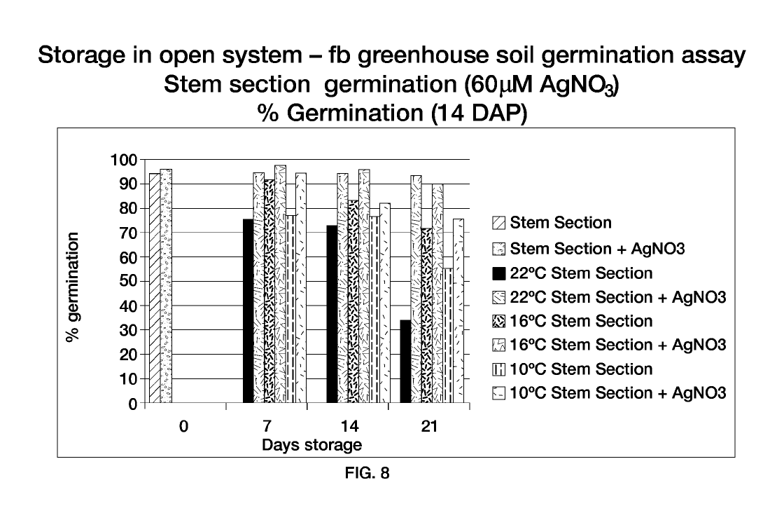

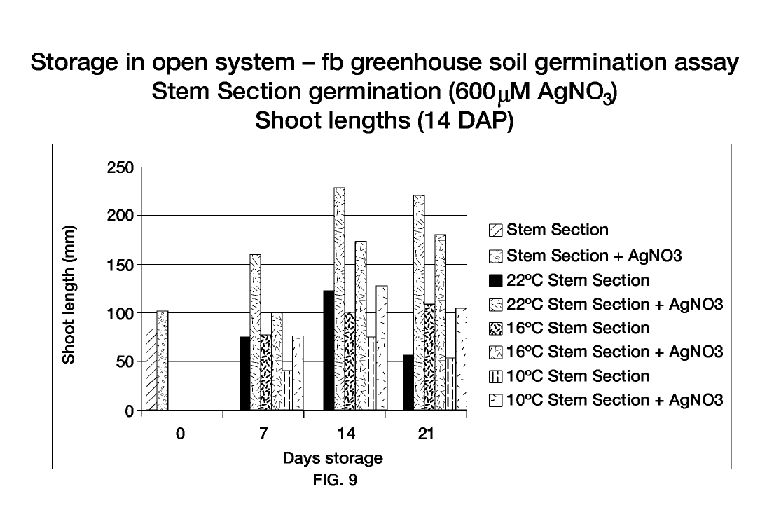

Results are shown in FIGS. 8-9. As shown in FIGS. 8-9, in all cases, the AgNO₃ treated sugarcane stem sections showed improved germination rates and plant vitality (as measured by plant height) when compared to control sugarcane stem sections (i.e., identical sugarcane stem sections that were treated identically as the AgNO₃ treated sugarcane stem sections except did not have AgNO₃ in the treatment slurry). In particular, the AgNO₃ treated sugarcane stem sections demonstrated significant enhancement of stem section germination at 7 through 21 days storage, and significant enhancement of shoot lengths at all storage periods including day 0 planting.

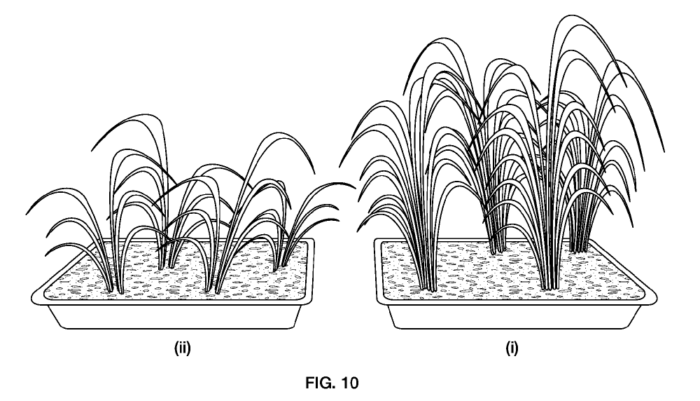

In addition, FIG. 10 depicts a comparison between (i) sugarcane plants formed via AgNO₃ treated sugarcane stem sections stored for 14 days in the above-described storage container at 22° C. and (ii) sugarcane plants formed via control sugarcane stem sections stored for 14 days in the above-described storage container at 22° C. All plants are shown 28 days after planting under the described greenhouse growing conditions. As shown in FIG. 10, sugarcane plants formed from AgNO₃ treated sugarcane stem sections demonstrated much more vitality when compared to sugarcane plants formed from control sugarcane stem sections without AgNO₃ treatment.

### EXPERIMENT 4

### Preparation, Extended Storage and Planting of AgNO₃ Treated Sugarcane Stem Sections

Sugarcane stem sections of CP88-762 having a diameter of approximately 25-30 mm and a length of approximately 50 mm were cut and treated with a standard treatment slurry or a standard treatment slurry containing AgNO₃ at a final concentration of 600 μMol of AgNO₃ as described in Example 3. The AgNO₃ treated and control (without AgNO₃) sugarcane stem sections were then planted as is or stored for an extended period of time up to 42 days in storage containers as shown at FIG. 1 (i.e., storage containers that allow gas transfer between an interior and so exterior of the storage container) at a storage temperature of 10° C. Following storage, the AgNO₃ treated and control sugarcane stem sections were planted in trays of potting soil and placed in a greenhouse as described in example 3. All sugarcane stem sections were evaluated for germination and shoot growth at 14 days after planting.

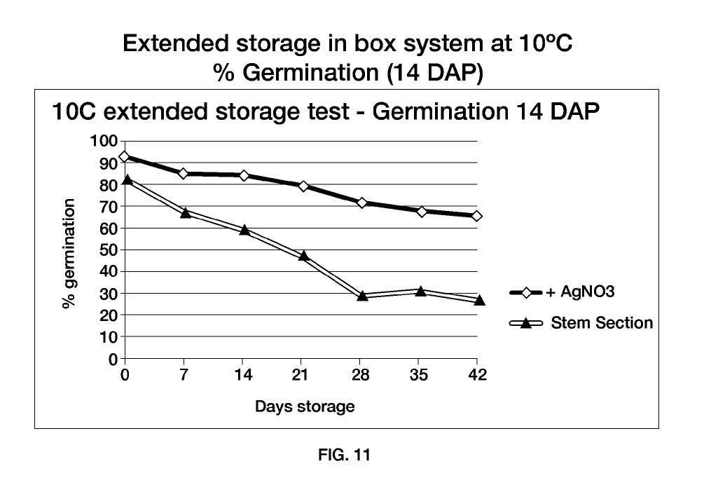

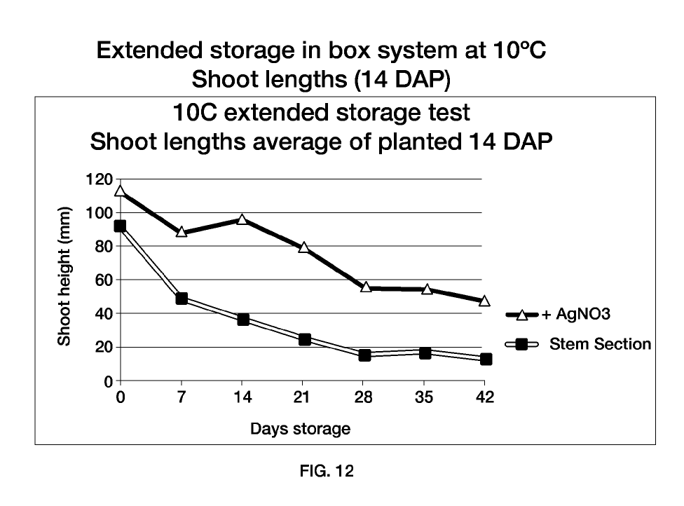

Results are shown in FIGS. 11-12. As shown in FIGS. 11-12, throughout the 42 days of storage, the AgNO₃ treated sugarcane stem sections showed improved germination rates and plant vitality (as measured by plant height) when compared to control sugarcane stem sections (i.e., identical sugarcane stem sections that were treated identically as the AgNO₃ treated sugarcane stem sections except did hot have AgNO₃ in the treatment slurry). At times during the 42 day storage period, the AgNO₃ treated sugarcane stem sections demonstrated germination rates greater than germination rates of control sugarcane stem sections (see, FIG. 11), and the resulting sugarcane plants produced via the AgNO₃ treated sugarcane stem sections demonstrated shoot heights greater than sugarcane plants produced vat the control sugarcane stem sections (see, FIG. 12). Further, as shown in FIG. 11, AgNO₃ treated sugarcane stem sections demonstrated a 68% germination rate with silver nitrate treatment after 6 weeks and storage at 10° C. which was substantially equivalent to the germination rate of control control sugarcane stem sections (i.e., without silver nitrate; alter 1 week of storage at 10° C.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed thereat, and every number between the end points. For example, a stated range of “1 to 10” should be considered to include any and all subranges between (and inclusive of) the minimum value of 1 and the maximum value of 10; that is all subranges beginning with a minimum value of 1 or more, e.g. 1 to 6.1, and ending with a maximum value of 10 or less, e.g., 5.5 to 10, as well as all ranges beginning and ending within the end points, e.g. 2 to 9, 3 to 8, 3 to 9, 4 to 7, and finally to each number 1, 2, 3, 4, 5, 6, 7, 8, 9 and 10 contained within the range.

It is further noted that, as used in this specification, the singular forms “a,” “an” and “the” include plural referents unless expressly and unequivocally limited to one referent.

It should be understood that although the above-described coated sugarcane stem sections, sugarcane plants resulting therefrom, and methods are described as “comprising” one or more components or steps, the above-described coated sugarcane stem sections, sugarcane plants resulting therefrom, and methods may “comprise,” “consists of,” or “consist essentially of” any of the above-described components, features or steps of the coated sugarcane stem sections, sugarcane plants resulting therefrom, and methods. Consequently, where the present invention, or a portion thereof has been described with an open-ended term such as “comprising,” it should be readily understood that (unless otherwise stated) the description of the present invention, or the portion thereof, should also be interpreted to describe the present invention, or a portion thereof using the terms “consisting essentially of” or “consisting of” or variations thereof as discussed below

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a coated sugarcane stem section and/or method that “comprises” a list of elements (e.g., components, features or steps) is not necessarily limited to only those elements for components or steps), but may include other elements for components or steps) not expressly listed or inherent to the covering and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements for components or steps) set forth in that clause; other elements for components) are not excluded from the claim as a whole

As used herein, the transitional phrases “consists essentially of” and “consisting essentially of” are used to define a coated sugarcane stem section, sugarcane plant resulting therefrom, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Further, it should be understood that herein-described coated sugarcane stem sections, sugarcane plants resulting therefrom, and methods may comprise, consist essentially of, or consist of any of the herein-described components and features, as shown in the figures with or without any feature(s) not shown in the figures. In other words, in some embodiments, the coated sugarcane stem sections, sugarcane plants resulting therefrom, and methods of the present invention do not have any additional features other than those shown in the figures, and such additional features, not shown in the figures, are specifically excluded from the coated sugarcane stem sections, sugarcane plants resulting therefrom, and methods. In other embodiments, the coated sugarcane stem sections, sugarcane plants resulting therefrom, and methods of the present invention do have one or more additional features that are not shown in the figures.

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

1. A method of extending shelf life and vitality of a sugarcane stem section, said method comprising:
applying to at least a portion of the sugarcane stem section, a composition comprising at least one additive selected from: silver nitrate, silver acetate, silver thiosulfate, potassium ferrocyanide, and potassium ferricyanide.

2-3. (canceled)

4. The method of claim 1, wherein the sugarcane stem section comprises one or more nodes along the sugarcane stem section.

5. The method of claim 1, wherein the sugarcane stem section comprises a single node along the sugarcane stem section.

6. The method of claim 1, wherein the composition comprises silver nitrate.

7. (canceled)

8. The method of claim 1, wherein the composition comprises one or more additives, wherein each additive is independently present at a concentration of from about 200 to about 1200 μMol.

9. The method of claim 8, wherein the composition comprises one or more additives, wherein each additive is independently present at a concentration of from about 300 to about 600 μMol.

10. (canceled)

11. The method of claim 1, wherein the composition further comprises one or more fungicides or one or more insecticides, or both.

12. (canceled)

13. The method of claim 1, wherein said application step comprises dipping the sugarcane stem section in the coating composition for a time period.

14. The method of claim 13, wherein the time period is less than one minute.

15-17. (canceled)

18. The method of claim 1, wherein the dried coated sugarcane stem section comprises from about 0.1 to 100 μMol of silver nitrate, silver acetate, silver thiosulfate, potassium ferrocyanide, or potassium ferricyanide, or a combination thereof.

19. The method of any one of claims 15 to 18, further comprising:
storing sugarcane stem section with dried coating for a storage time period from about seven days up to six weeks in a storage container at a storage temperature.

20-27. (canceled)

28. A sugarcane stem section formed by the method of claim 1.

29. (canceled)

30. A method of growing sugarcane plants comprising:
planting one or more sugarcane stem sections formed by the method of claim 1 in a growing medium.

31. A method of increasing vitality of sugarcane plants formed from sugarcane stem sections, said method comprising:
forming sugarcane stem sections using the method of claim 1; and
planting one or more sugarcane stem sections formed by the method claim 1 in a growing medium.

32-37. (canceled)

38. The method of claim 1, wherein the additive is present on the sugarcane stem section in amount from 0.01 to 100 μMol.

39. The method of claim 1, wherein the additive is present on the sugarcane stem section in amount from 0.1 to 1.2 μMol.

40. The method of claim 1, wherein the additive is present on the sugarcane stem section in amount from 0.08 to 0.3 μMol.
